(19) United States
(12) Patent Application Publication
(10) Pub. No.: US 2026/0298900 A1
(43) Pub. Date: Oct. 1, 2026

(54) APPARATUS FOR TESTING ELECTRONIC SEMICONDUCTOR COMPONENTS

(71) Applicant: Uwe BEIER, Ottendorf-Okrilla (DE)

(72) Inventor: Uwe BEIER, Ottendorf-Okrilla (DE)

(21) Appl. No.: 19/132,558

(22) PCT Filed: Nov. 27, 2023
(86) PCT No.: PCT/DE2023/100923

§ 371 Date: May 23, 2025

(30) Foreign Application Priority Data
Nov. 29, 2022 (DE) 102022131475.8

(51) Int. Cl. G01N 33/00; G01R 31/28

(52) U.S. Cl. CPC G01N 33/0072; G01N 33/0027; G01R 31/2862; G01R 31/2863; G01R 31/2865

(57) ABSTRACT

An apparatus for testing an electronic semiconductor component which has a sensor layer for detecting a gaseous substance and is formed on a substrate using a test gas. The apparatus has a chamber enclosing an interior in which negative or positive pressure prevails and in which there are arranged a holding device for holding the substrate; and a nozzle for directionally delivering the test gas onto the sensor layer of the electronic semiconductor component formed on the substrate held by the holding device.

### FIELD

The invention relates to an apparatus for testing electronic semiconductor components. Furthermore, it relates to a method for testing electronic semiconductor components.

### BACKGROUND

Electronic semiconductor components having a sensor layer for the detection of a gaseous substance can be used for detection of gaseous substances. If the gaseous substance contains a certain constituent an electric signal is generated by means of the electronic semiconductor. For contacting the electronic semiconductor components in addition to the sensor layer they have a contact surface for electrically contacting the electronic semiconductor component. Said contact surfaces are also referred to as contact pads. Such electronic semiconductor components are used as sensors used for a plurality of different purposes, for example to localize leakage. In particular, they can be used to analyze the chemical composition of gases. Such sensors are also referred to as gas sensors or chemical gas sensors.

Such electronic semiconductor components can be prepared on the basis of the semiconductor technology. For that, thin disks, so-called wafers, are used as the base substrates and provided with sensitive layers in a plurality of process steps so as to gradually build up electronic semiconductor components. The precursors prepared until the finished electronic semiconductor components are obtained themselves also represent electronic semiconductor components.

The disks may be for example disks of silicon, ceramics, or glass. The sensitive layers are applied in such a way that a large number of electronic semiconductor components can be formed on one wafer. The electronic semiconductor components are usually small compared to the size of the wafer in the surface direction. In this way, it can be provided for a sufficient productivity, because in each of the often complex process steps for manufacturing the electronic semiconductor components, several of them are processed simultaneously. Nevertheless, the individual process steps are complex and therefore cost-intensive.

It is therefore advantageous that between the individual process steps and after completion of the electronic semiconductor components, i.e. before the cost-intensive dicing, i.e. separation, and packaging, i.e. encapsulation, each precursor and the finished electronic semiconductor component are checked for function, in order to be able to exclude the defective electronic semiconductor component directly. Defective electronic semiconductor components can then be extracted.

To test the precursors and the finished electronic semiconductor components, these are contacted at their contact surfaces with special needles and fully operated via downstream electronics. Depending on the type of electronic semiconductor components to be manufactured, it may be useful or necessary to carry out these tests in a vacuum environment. There are sufficient examples of this in the state of the art.

The rapid digitization and networking of digital components, described under keywords such as “Internet of Things” (“IoT”) and Industry 4.0, require an increasing number of sensors in order to grasp the current conditions at various points and to be able to react accordingly.

EP 3 486 639 B1 describes an apparatus, referred to there as a prober, which shows the basic structure of a test device that can be used for testing electronic semiconductor components, which however are not gas sensors.

To be able to test finished electronic semiconductor components that are to be used as gas sensors and their precursors on the substrate, i.e. before separation, according to the prior art they are placed in a test environment which is completely filled with a test gas. Since it is usually necessary to move the substrates to individually contact each electronic semiconductor component, such test environments require space for mechatronic moving and contacting systems. The test devices in which such test environments are designed therefore require a lot of space.

If the entire test environment is now flooded with the test gas, i.e. the fluid to be detected, in the required composition on the one hand large quantities of test gas are required compared to the size of the sensor layer of an electronic semiconductor component. On the other hand- and this is the much bigger problem-this test gas of course also reaches all surfaces present within the test environment, for example surfaces of other elements, components, and materials, and deposits there. The test gas or a component of the test gas can penetrate the element, component, or material. The deposition as well as the penetration can lead to changes in the properties of the elements, components, and materials, but at least this significantly decelerates the test device and often leads to drift, i.e. to a slow change in the initial position. If aggressive gases are used as test gases damages to the surrounding components are also possible. It is possible to counteract this deposition and penetration by means of special test gas pumping techniques. However, the pumping techniques must be sized large and require additional long pumping times, which means time and energy expenditure and thus costs and reduces the effectiveness of testing and thus manufacturing of the electronic semiconductor components.

It is the problem of the invention to eliminate the drawbacks according to the prior art. In particular, an apparatus for testing electronic semiconductor components is to be provided which requires a smaller quantity of test gas compared to the prior art.

### SUMMARY

According to the invention an apparatus for testing an electronic semiconductor component is provided which has a sensor layer for detecting a gaseous substance and is formed on a substrate using a test gas. In this case, the apparatus has a chamber enclosing an interior in which negative or positive pressure prevails and in which there are arranged
a holding device for holding the substrate; and
a nozzle for directionally delivering the test gas onto the sensor layer of the electronic semiconductor component formed on the substrate held by the holding device.

The directed delivery of the test gas onto the sensor layer of the semiconductor component allows the use of a small amount of test gas. When using the apparatus according to the invention it is not required to flood the entire test environment, i.e., the entire interior of the apparatus, with the test gas. Rather, it is sufficient that only the sensor layer comes into contact with the test gas. According to the invention, this is ensured by directing a stream of the test gas onto the sensor layer of the electronic semiconductor component by means of the nozzle. The electronic semiconductor component is held on the holding device. By means of the apparatus according to the invention, a significant increase in efficiency and measurement accuracy is achieved in the testing of semiconductor components that are to be used as gas sensors. In addition, an increase in flexibility is achieved if the chemical composition of the test gas is to be changed. The deposition of test gas on elements, components, and/or materials of the apparatus according to the invention and thus the drift are reduced. The test gas reaches the sensor surface and from there on into the interior of the chamber. It can then be extracted from the chamber via an outlet. The contact time of the test gas with elements, components, and/or materials other than the substrate carrying the electronic semiconductor component is therefore short. For example, a pump can be provided for extract-ing the test gas from the interior of the chamber.

The substrate may be a wafer, for example a wafer made of silicon, ceramics, or glass. The electronic semiconductor component which has a sensor layer for detecting a gaseous substance is formed on the substrate. A plurality of such electronic semiconductor components can be formed on the substrate. The substrate has a top side on which the electronic semiconductor component is formed and a bottom side. The electronic semiconductor component has a sensor layer which is spaced from the boundaries of the electronic semiconductor components to form an area surrounding the sensor layer. The electronic semiconductor component also has contact surfaces for contacting by a contact element. Preferably, the contact surfaces are spaced apart from the boundaries of the electronic semiconductor component and the sensor layer. Preferably, the contact surfaces are located outside of the surrounding area. They can be arranged circumferentially around the sensor layer.

When using the apparatus according to the invention for testing an electronic semiconductor component negative pressure or positive pressure prevails in the interior. During testing a pressure should prevail in the interior that deviates from the ambient pressure. Said pressure is also referred to as chamber pressure. Preferably, negative pressure prevails in the interior. For example, a negative pressure of 10⁻⁵ mbar or less can prevail in the interior. The test gas should be gaseous at the chamber pressure. One or more devices for supplying the test gas to the nozzle may be provided for supplying the test gas to the nozzle. The test gas can be supplied to the sensor layer of the held semiconductor component at a pressure referred to as the test pressure. Preferably, the test pressure is above the chamber pressure. It can be controlled independently of the chamber pressure.

The test gas can be produced in the nozzle itself. For that, components of the test gas to be produced can separately be supplied to the nozzle. For example, the test gas can be produced in the nozzle from a first gas and a second gas. Both gases each can be supplied to the nozzle by means of one or more devices for supplying a gas.

The test gas can be a mixture of several gases. The test gas can contain the gaseous substance, but this is not mandatory. The gaseous substance is the substance that is to be detected by the sensor layer.

Preferably, the nozzle has a first nozzle opening through which the test gas can escape from the nozzle. It may be provided that the nozzle is configured to discharge a sealing gas or to receive the test gas. For that, the nozzle can have a second nozzle opening in addition to the first nozzle opening. The sealing gas can escape from the nozzle through the second nozzle opening or the test gas can enter the nozzle again after contact with the sensor layer. The second nozzle opening can be coaxial with the first nozzle opening. The second nozzle opening can be formed circumferentially around the first nozzle opening. The second nozzle opening can be separated from the first nozzle opening by a nozzle web. The nozzle can have further nozzle openings, for example a third nozzle opening. These further nozzle openings can also be formed coaxially to the first nozzle opening and to the second nozzle opening. They can each serve to release a further gas or to receive a gas or gas mixture. Preferably, the nozzle has a nozzle body in which the first nozzle opening is formed. The second nozzle opening can also be formed in the nozzle body. In one embodiment, the nozzle body has at least one port via which the test gas can be supplied to the nozzle. A gas can be supplied to this port by means of the devices for supplying a gas to the nozzle. The nozzle body can only have one port via which the test gas is supplied to the nozzle. Alternatively, the nozzle body can have exactly two ports. The first port can be used to supply the test gas and the second port can be unused. If the test gas in the nozzle is to be produced from a first gas and a second gas, the first port can be used to supply the first gas and the second port can be used to supply the second gas. The nozzle body can have a further port via which a sealing gas can be supplied to the nozzle.

The sealing gas can be supplied to the held semiconductor component at a pressure known as the sealing gas pressure. Preferably, the sealing gas pressure is above the chamber pressure. It can be controlled independently of the chamber pressure. The physical states of the sealing gas can differ from those of the test gas. For example, this relates to one or more of the following properties: the gas composition, the pressure, the temperature, the humidity, the flow rate, the volume flow.

The nozzle body can be a symmetrical nozzle body with respect to its outer shape. However, it can be a symmetrical nozzle body as a whole, i.e., not only with respect to its outer shape. The axis of symmetry can be its longitudinal axis. A symmetrical nozzle body can have two ports, one of which is used to supply the test gas or, if the test gas is to be produced from a first gas and a second gas in the nozzle, to supply the first gas. The second port can be unused if the test gas is supplied via the first port. If the test gas is to be produced from a first gas and a second gas in the nozzle, it can be used to supply the second gas. In order to enable the mixing of the first gas and the second gas to form the test gas, a nozzle insert can be formed in the nozzle body. The nozzle insert can have one or more elements for guiding the first gas and/or the second gas.

The material the nozzle body is made from can be selected from different materials. Depending on the material, the nozzle can be adapted to different test conditions, for example different test gases, test pressures, and/or chamber pressures.

According to the invention a holding device is provided for holding the substrate. Preferably, the holding device is configured such that the substrate with its bottom side rests on the holding device. For that, the holding device can have a holding surface. The upper side of the substrate with the electronic semiconductor component faces away from the holding surface and is therefore exposed. This means that the sensor layer of the electronic semiconductor component is also exposed. If the substrate on which the electronic semiconductor component is arranged is held by the holding device, the electronic semiconductor component is also referred to as a held semiconductor component.

The nozzle provided according to the invention is for the directed delivery of the first test gas onto the sensor layer of the electronic semiconductor component. In order to enable the directed delivery of the first test gas, the nozzle is preferably arranged opposite the holding device and at a distance from the latter. The distance between the nozzle and the holding device can preferably be formed in such a way that a distance is formed between the nozzle and the holding device when the holding device holds the substrate. In other words, this means that the nozzle preferably touches neither the held semiconductor component nor the substrate. However, in one embodiment, it may be provided that the nozzle touches the held semiconductor component or the substrate, even if this is not preferred. Unwanted contact between the nozzle on the one hand and the held semiconductor component or the held substrate on the other hand can result in damage to the held substrate and/or the held semiconductor component and contamination of the held substrate and/or the held semiconductor component with material elements of the nozzle. Therefore, in most cases, contact between the nozzle and the held semiconductor component or the held substrate has mandatory to be avoided.

If the holding device holds the substrate, it may be provided that the first nozzle opening is arranged opposite the held semiconductor component. If the holding device holds the substrate and the nozzle has a second nozzle opening it may be provided that the first nozzle opening and the second nozzle opening are arranged opposite the held semiconductor component.

Preferably, the nozzle body has a first front face that faces the holding device. The first nozzle opening is formed in the first front face. If the nozzle has a second nozzle opening the second nozzle opening is preferably also formed in the first front face of the nozzle body. The distance in the height direction between the first front face of the nozzle body and the sensor layer of the held semiconductor component should be as small as possible. Preferably, the distance in the height direction between the first front face of the nozzle body and the sensor layer of the held semiconductor component is greater than 0 and less than 0.1 mm.

The nozzle body can have a second front face opposite the first front face. The channel through which the test gas is guided to the first nozzle opening can extend between the first and second front faces. A window can be formed in the second front face. The window allows observation of the held semiconductor component. An optical device, for example a microscope, can be provided for observation. The optical device can be arranged outside the chamber. The window can be formed in the center of the second front face. The positioning of the held semiconductor component can be ob-served through the window, the adjoining channel and the first nozzle opening. In particular, the positioning of the held semiconductor component under the contacting device and/or the nozzle can be observed. Observation is possible from above.

It may be provided that the first nozzle opening is aligned with the sensor layer of the held semiconductor component. The term aligned refers to an axis G which is orthogonal to the first nozzle opening and to the surface side of the sensor layer facing the first nozzle opening. Preferably, the axis G is orthogonal to the holding surface of the holding device. Then, the first nozzle opening is aligned with the holding surface. Preferably, the geometric shape of the nozzle opening is adapted to the geometric shape of the electronic semiconductor component and/or to the geometric shape of the sensor layer of the electronic semiconductor component. It may be provided that the first nozzle opening has a rectangular shape. This is useful because most electronic semiconductor components are rectangular for reasons of space utilization. In addition, the dimensions of the first nozzle opening, namely in a plane parallel to the surface direction of the sensor layer, can also be adapted to the dimensions of the electronic semiconductor component and/or the dimensions of the sensor layer of the electronic semiconductor component. In this context, the term “adapted” is intended to express that the test gas stream is directed only towards the held semiconductor component or only towards its sensor layer and an area surrounding the sensor layer.

It may be provided that the second nozzle opening is formed opposite the circumferential area of the held semiconductor component. In this way, the sealing gas can be guided to the held semiconductor component. The sealing gas is preferably an inert gas, for example argon or nitrogen. The sealing gas should not contain the gaseous substance that is to be detected by the sensor layer. The sealing gas can provide a barrier function against the diffusion of ambient gas. For that, it is advantageous that the test gas with a test pressure and the sealing gas with a sealing gas pressure, both of which are above the chamber pressure, flow out of the gap between the first front face of the nozzle and the held semiconductor component. This shields the sensor layer to which the test gas is directed from the ambient gas.

The semiconductor component can have a contact surface which is spaced from the sensor layer to form a spacer surface with the second nozzle opening being opposite the spacer surface.

Preferably, the nozzle tapers toward the holding device. In addition, it may be provided that the nozzle has a nozzle wall whose material thickness decreases toward the holding device.

The nozzle can have a nozzle body in which a first channel is formed through which the test gas flows to the first nozzle opening. Preferably, the nozzle body is gas-tight to be able to guide the test gas without leakage. A second channel can be formed in the nozzle body through which the sealing gas flows to the second nozzle opening or through which the test gas received via the second nozzle opening is led off. A nozzle web can be formed in the nozzle body which separates the first channel from the second channel.

The recirculation of the test gas via the second nozzle opening has various advantages. The recirculation of the test gas can enable a detailed analysis of the test gas which has passed the held semiconductor component. For that, the apparatus according to the invention can have a gas analyzer. The gas analyzer can be arranged in a line that is connected to the nozzle to withdraw the test gas. In addition, the recirculation of the test gas via the second nozzle opening also acts as a barrier against the diffusion of ambient gas from the interior of the chamber. In addition to the test gas, ambient gas is also withdrawn through the second nozzle opening. The common withdrawal of test gas and ambient gas generates a pressure sink over the test gas flowing through the gap between the nozzle and the held semiconductor component at a slightly higher pressure than the chamber pressure. Therefore, the sensor layer is shielded from the ambient gas by recirculating the test gas via the second nozzle opening. Here, the ambient gas is the gas that is inside the chamber, apart from the test gas and, if provided, the sealing gas, as long as these have not escaped from the gap between the first front face of the nozzle and the held semiconductor component.

The apparatus according to the invention can have a device for displaying the distance and/or for measuring the distance between the held semiconductor component, for example the sensor layer, and the nozzle. In particular, it can have a device for displaying the distance and/or for measuring the distance between the held semiconductor component, for example the sensor layer, and the first front face of the nozzle. The device may be a height indicator or a measuring device mounted to the nozzle. The measuring device can be a test needle, for example. For example, it may be provided that the movement of the nozzle towards the held semiconductor component ends when the test needle touches a contact surface of the held semiconductor component. The construction of the test needle can correspond to the construction of a contact needle of the contacting device. However, the measuring device can also be a sensor, for example a capacitive sensor, an inductive sensor or another sensor, which are suitable for the stated purpose according to the prior art.

The apparatus according to the invention may further comprise a positioning device for positioning the nozzle. By means of the positioning device the nozzle can be held and moved. By means of the positioning device the nozzle can be positioned opposite the sensor layer of the held semiconductor component. By means of this positioning device the nozzle can be moved in the x, y and z directions and rotated about an axis lying on the z coordinate. The height positioning of the nozzle, i.e., the positioning with respect to the z coordinate, is of particular importance. Alternatively or addition-ally, the apparatus according to the invention can have a positioning device for positioning the holding device. By means of this positioning device the holding device can be movable in the x, y and z directions and rotatable about an axis lying on the z coordinate. The positioning device can be a manipulator. If no positioning device is provided for the nozzle the nozzle can alternatively be held by the contacting device described below.

The apparatus according to the invention can have a contacting device for the electrical contacting of the held semiconductor component at its contact surface. The contacting device can be a so-called probe card. The contacting device can have an opening through which the nozzle is guided. Alternatively, the nozzle can be firmly connected to the contacting device.

The contacting device has contact needles with which it contacts the contact surfaces of the held semiconductor component. It is therefore advantageous if the nozzle body is as thin-walled as possible on its first front face which faces the held semiconductor component. For that, it can be provided that the nozzle body tapers towards its first front face and for this purpose its material thickness reduces. In this way, it can be ensured that sufficient space is left for the contact pins of the contacting device without touching them and, at the same time, that alignment movements of the contact pins are possible. An increase in the material thickness of the nozzle body, starting from its first front face toward its opposite second front face, is advantageous to achieve stabi-lization of the nozzle and -at a sufficient distance from the first front face-to be able to attach suitable, preferably standardized fastening elements to the supporting structure of the nozzle.

The nozzle can be firmly connected to the contacting device, for example via one or more connecting elements. The nozzle is preferably in an adjusted position with respect to the contacting device. In this way, a collision between the nozzle and a contact needle can be prevented. Alignment of the nozzle by means of a positioning device is not necessary. Also, a separate device for holding the nozzle is not necessary. How-ever, the production of the contacting device is more complex.

The nozzle may have one or more portsvia which the test gas, components of the test gas, or the sealing gas are separately supplied to the nozzle. A line can be provided for guiding each of the test gas, its components, and the sealing gas to a port. The connection point between a port and a line is preferably sealed, for example with a sealing system that is suitable for use under negative pressure and/or positive pressure.

The apparatus according to the invention can have one or more devices for ad-justing the chamber pressure. In particular, it can have one or more devices for generating negative pressure or positive pressure in the chamber. The device for generating negative pressure can be a pump. The pump can be arranged outside the chamber. An opening serving as outlet can be provided in the chamber wall from which the gas located in the interior of the chamber can be withdrawn via a line by means of the pump.

The apparatus according to the invention can have one or more devices for guiding the test gas, the first gas, and/or the second gas to the nozzle. For example, the test gas can be guided to the nozzle via one or more lines by means of a pump. The apparatus according to the invention can have one or more devices for guiding the sealing gas to the nozzle. For example, the sealing gas can be guided to the nozzle via one or more lines by means of a pump. The apparatus according to the invention can have one or more devices for leading off the test gas from the nozzle. For example, the test gas can be led off from the nozzle via one or more lines by means of a pump. Such a pump may be an exhaust pump.

The apparatus according to the invention permits a targeted guiding of the test gas and, if provided, the sealing gas. Only very small amounts of gas are required. For this reason, the apparatus according to the invention permits a very rapid change in process parameters, for example the test gas flow, the chamber pressure, the test pressure, the concentration of a component of the test gas, and the sealing gas flow, with the list not being exhaustive. The apparatus according to the invention permits the rapid testing of a large number of electronic semiconductor components, also under different test conditions.

The first nozzle opening is formed such that the stream of the test gas which escapes from the first nozzle opening is only directed to one of the electronic semiconductor components, namely to the electronic semiconductor component to be tested. Electronic semiconductor components adjoining the electronic semiconductor component to be tested on the substrate are not or only very slightly pressurized with the test gas. This is of decisive advantage for many applications compared to the prior art according to which all electronic semiconductor components formed on a substrate come into contact with the test gas within the chamber over the whole time.

By means of the device for adjusting the chamber pressure it can be ensured that the concentration of test gas in the interior of the chamber does not rise above or fall below a given level. By means of the device for adjusting the chamber pressure the test gas which escapes at the first nozzle opening of the nozzle and contacts the held semiconductor component can be withdrawn. The device for adjusting the chamber pressure preferably is a pump. The test gas escaping from the nozzle is immediately evacuated by the pump after it has contacted the sensor layer of the held semiconductor component. That is, the concentration of the test gas in the entire chamber does not change so that contaminations and damages to devices within the chamber, for example to mechatronic systems such as e.g., positioning devices, are prevented. This is a major advantage.

By means of the apparatus according to the invention, comparative measurements and calibrations can be carried out in a simple manner using measuring devices known from the prior art. If there is negative pressure in the chamber, the measuring device can be a vacuum measuring device. For example, a so-called residual gas analyzer (“RGA”) can be attached to the chamber. Measurements can be carried out under different pressures. A certain pressure is referred to as the pressure level. Different pressures can be easily set using the apparatus according to the invention. For this purpose, the apparatus according to the invention can have an inlet that enables the supply of a gas via an opening in the chamber wall. The gas can, for example, be an ambient gas, e.g. ambient air, or a protective gas such as nitrogen or argon. The inlet can have one or more control devices, for example a valve.

According to the invention further provided is a method for testing an electronic semiconductor component which has a sensor layer for detecting a gaseous substance and is formed on a substrate, using a test gas by means of an apparatus which has a chamber enclosing an interior in which negative or positive pressure prevails, comprising
positioning a nozzle with respect to the sensor layer of the electronic semiconductor component to enable a directional delivery of the test gas onto the sensor layer;
contacting contact surfaces of the electronic semiconductor component by means of a contacting device; and
outputting a test signal at contact surfaces of the electronic semiconductor component by means of the contacting device, while the test gas is delivered to the sensor layer by means of the nozzle.

The method according to the invention can be carried out by means of the apparatus according to the invention. The method according to the invention may further comprise receiving a signal from the semiconductor component. Said signal can be received via contact surfaces of the semiconductor component by means of the contacting device. The signal received then can be compared to comparison values to determine whether the semiconductor component is defective or not.

Details of the method according to the invention have already been explained in the context of the apparatus according to the invention. Reference is made to said ex-planations.

### BRIEF DESCRIPTION OF THE FIGURES

In the following the invention is explained in more detail with the help of examples not intended to limit the invention with respect to the drawings. Here,

FIG. 1 shows a schematic plan view to a substrate from which a plurality of electronic semiconductor components is formed;

FIG. 2 shows a schematic plan view to one of the electronic semiconductor components shown in FIG. 1;

FIG. 3 shows a schematic illustration of a first embodiment of an apparatus according to the invention;

FIG. 4 shows a schematic plan view to a holding device with a substrate lying thereon;

FIG. 5 shows a schematic plan view to a held semiconductor component;

FIG. 6 shows a schematic illustration of a first nozzle of the first embodiment of the apparatus according to the invention together with the substrate held by the holding device with the holding device not being shown;

FIG. 6A shows a schematic plan view to the nozzle from the first front face of the nozzle body;

FIG. 6B shows a schematic illustration of a part of the nozzle;

FIG. 6C shows a schematic illustration of the first nozzle of the first embodiment of the apparatus according to the invention together with the substrate held by the holding device with the holding device not being shown, wherein two gases are supplied to the nozzle;

FIG. 7 shows a detailed view of the first embodiment of an apparatus according to the invention;

FIG. 8 shows a schematic illustration for illustrating the size of the first nozzle opening;

FIG. 9 shows a schematic illustration of a second nozzle together with the substrate held by the holding device with the holding device not being shown;

FIG. 9A shows a schematic plan view to the second nozzle from the first front face of the nozzle body;

FIG. 9B shows a schematic illustration for illustrating the size of the first nozzle opening and the size of the second nozzle opening;

FIG. 10 shows an illustration of a second nozzle having a second nozzle opening which is for delivering a sealing gas;

FIG. 11 shows an illustration of the second nozzle, wherein the second nozzle opening is for receiving the test gas;

FIG. 12 shows a schematic illustration of a second embodiment of an apparatus according to the invention;

FIG. 13A shows a schematic illustration of a device for determining the distance between the nozzle and the held semiconductor component with the device being shown before the contact with the held semiconductor component; and

FIG. 13B shows a schematic illustration of the device for determining the distance between the nozzle and the held semiconductor component shown in FIG. 13A with the device being shown upon contact with the held semiconductor component.

### DETAILED DESCRIPTION

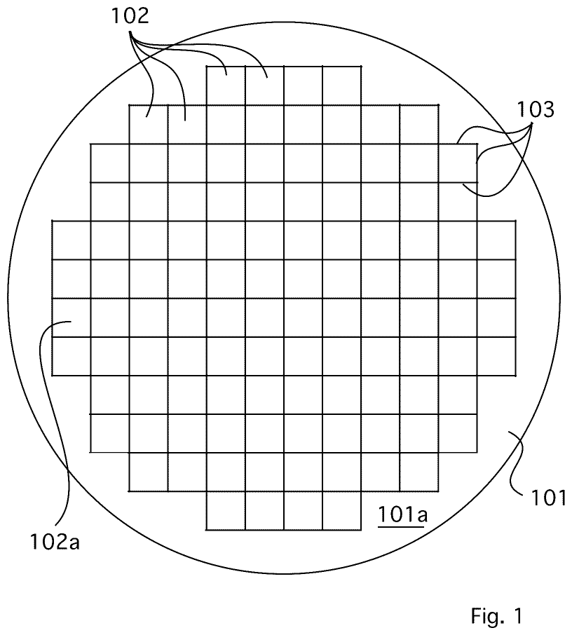

The x, y and z coordinates given in the figures refer to a Cartesian coordinate system. In FIG. 1, an exemplary substrate 101 is shown, on the upper side 101a of which a plurality of electronic semiconductor components 102, 102a is formed, which are separated from each other at the boundaries 103. The semiconductor components 102, 102a generally have the same construction, even if this is not mandatory. The electronic semiconductor components 102, 102a have a rectangular shape which is determined by the boundaries 103.

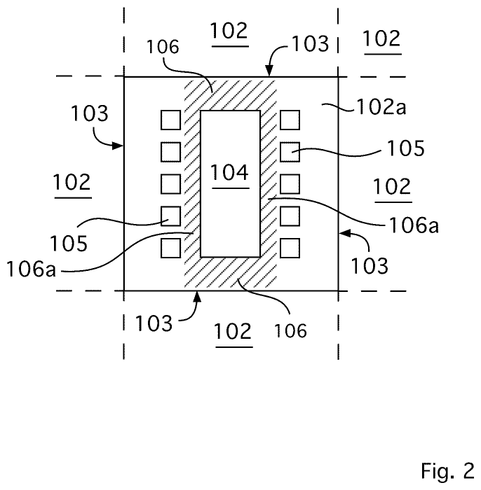

An exemplary electronic semiconductor component 102a which is arranged on the substrate 101 is shown in FIG. 2. The semiconductor component 102a has a sensor layer 104 for detecting a gaseous substance and contact surfaces 105 for electrical contacting. The sensor layer 104 is formed at a distance from the boundaries 103 of the semiconductor component 102a and the contact surfaces 105. The spacer surfaces 106 are located between the sensor layer 104 and the boundaries 103. In addition, the contact surfaces 105 are spaced apart from the boundaries. Spacer areas 106a which are part of the spacer areas 106 are formed between the contact surfaces 105 and the sensor layer 104. Thus, a circumferential area 107 which is separated by the boundaries of the semiconductor component 102a and the contact surfaces 105 and which is part of the spacer areas 106 adjoins the sensor layer 104. The surrounding area 107 is shown hatched in FIG. 2. The sensor layer 104 has a rectangular shape.

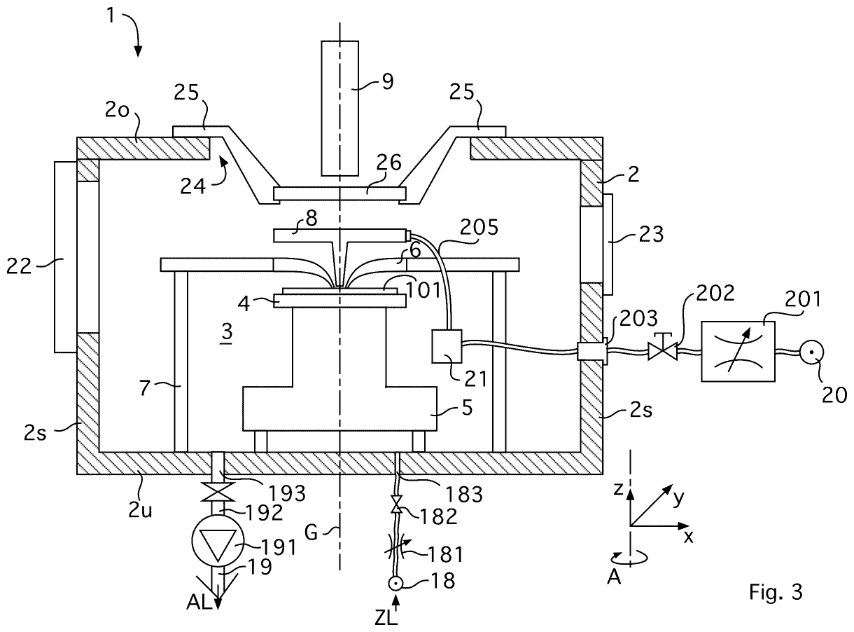

The first embodiment of an apparatus 1 according to the invention shown in FIG. 3 has a chamber 2 which encloses an interior 3. The chamber 2 has chamber walls, namely a bottom 2u, side walls 2s and a ceiling 2o. The side walls 2s are firmly connected to the bottom 2u. The ceiling 2o is formed as a removable lid which can be detachably attached to the side walls 2s. The contact surfaces between the side walls 2s and the lid are sealed by means of one or more sealing elements.

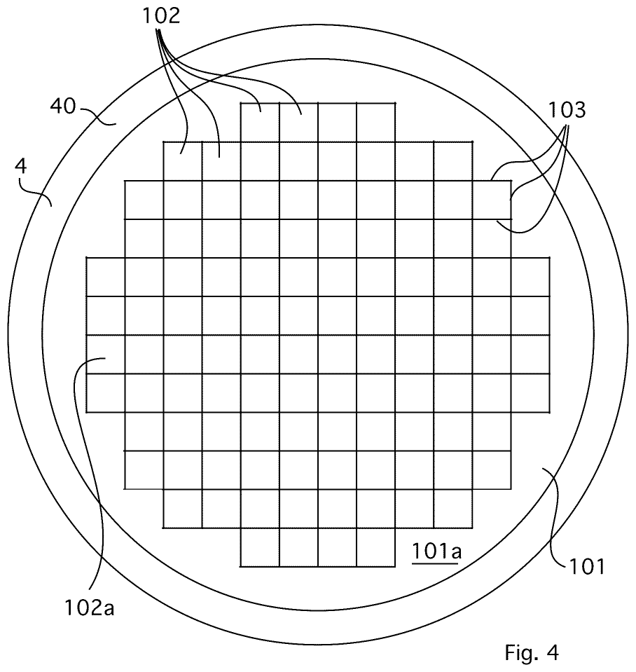

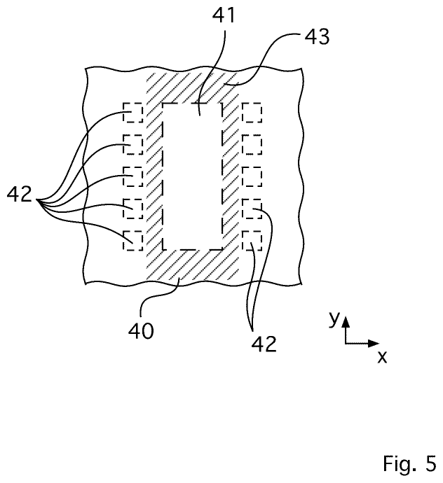

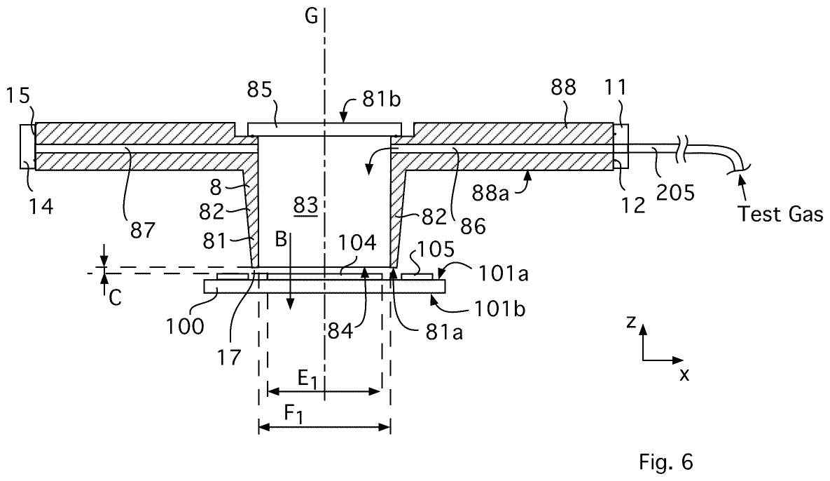

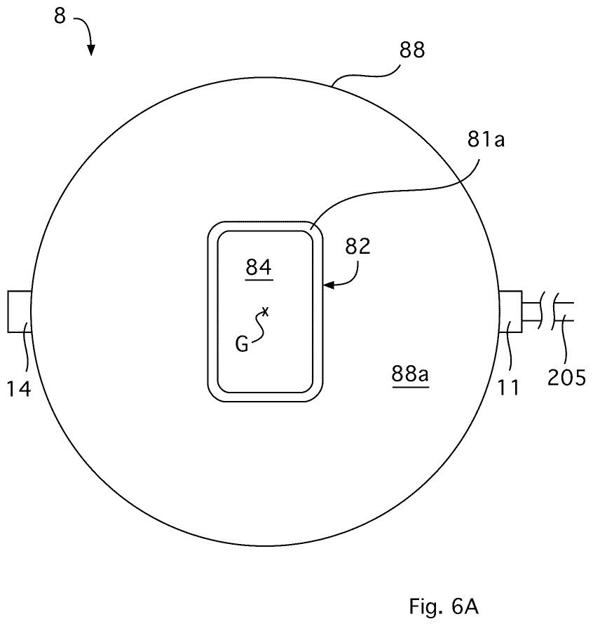

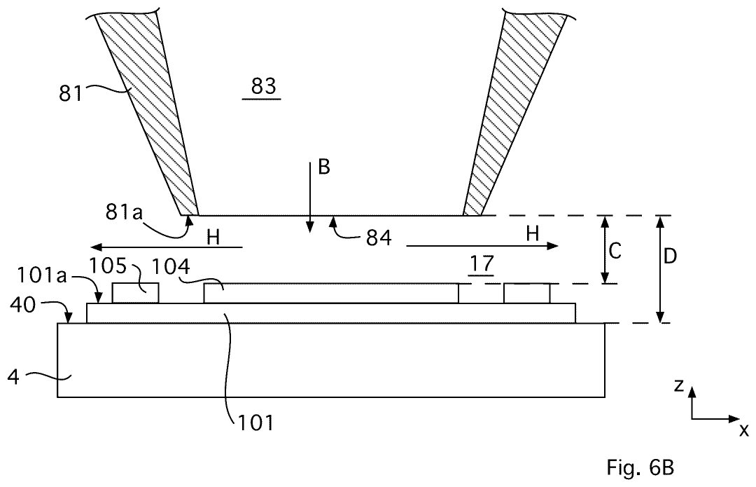

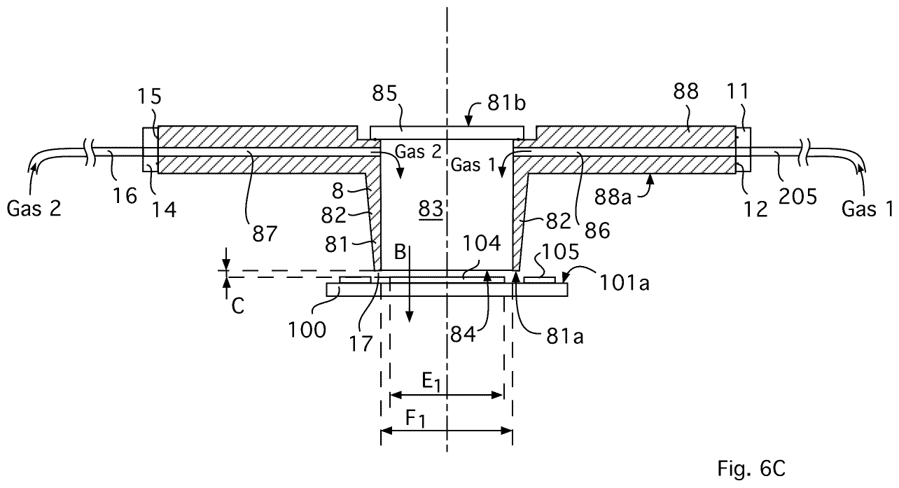

Negative or positive pressure can prevail in the interior 3. A holding device 4 for holding the substrate 101 is arranged in the interior 3. The holding device 4 is also referred to as a chuck. The holding device 4 has a holding surface 40 on which the substrate 101 rests with its bottom side 101b (see FIGS. 4 and 6). The exemplary semiconductor component 102a rests in a known position on the holding surface 40. A first substrate section of the substrate 101, on which the sensor layer 104 of the semiconductor component 102a is formed, rests on a first holding area 41 of the holding surface 40. Second substrate sections on which the spacer areas 106 are formed lie on second holding areas 42 of the holding surface 40. FIG. 5 shows a section of the holding surface 40 on which the semiconductor component 102a is held, wherein the first holding area 41 and the second holding area 42 are indicated by dashed lines. The hatched third holding area 43 which surrounds the first holding area 41 is the area in which a third substrate section of the substrate 101 on which the surrounding area 107 of the semiconductor component 102a is located rests on the holding surface 40. The holding device 4 is arranged on a positioning device 5 referred to as a stage, with which the holding device 4 can be moved in the x, y and z directions and can be rotated about an axis lying on the z axis. By means of the positioning device 5 the holding surface 40 of the holding device 4 can be positioned in such a way that the longitudinal axis of the first holding area lies on the G axis. The holding surface is also used to position the held semiconductor component so that the longitudinal axis of the held semiconductor component lies on the axis G. The term longitudinal axis refers to the z coordinate.

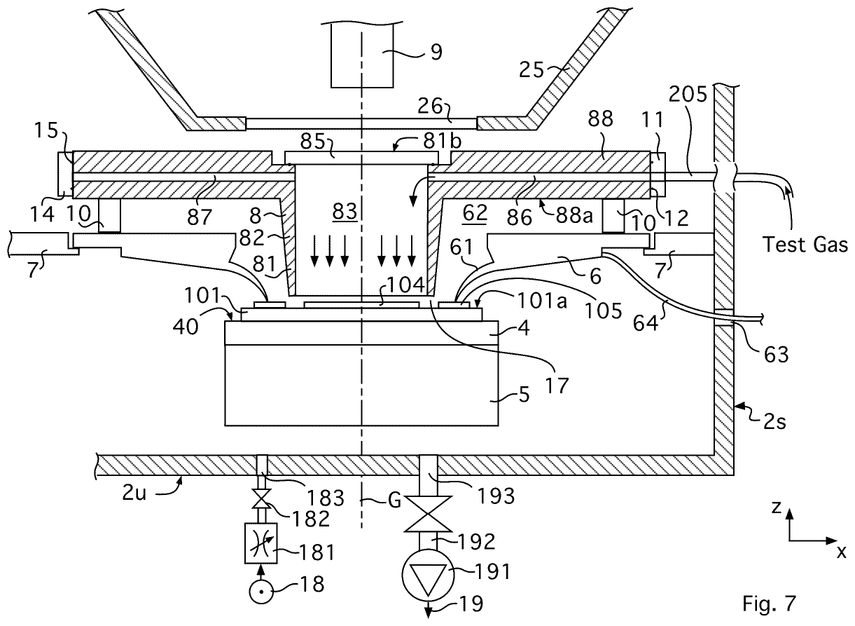

The interior 3 also contains a contacting device 6 for making electrical contact between the semiconductor component at the contact surfaces 105. The contacting device may be a probe card. The contacting device 6 is held by a holder 7. The contacting device has contact pins 61 (see FIG. 7). The contacting device 6 is opposite the holding surface 40 of the holding device 4. If the substrate 101 is held by the holding device 4 the contact needles 61 can be aligned with respect to the contact surfaces 105 of the semiconductor component 102 in such a way that the contact needles 61 come into electrical contact with the contact surfaces 105. An opening 62 (see FIG. 7) is formed in the contacting device 6, which—according to the prior art, as described for example in EP 3 486 639 B1 in the context of FIG. 5 there—enables observation of the semiconductor component 102 by means of an optical device 9 which is arranged outside the chamber 2. The contacting device 6 is supplied with electrical current via an electrical cable 64. In addition, signals can be sent to or received from the contacting device 6 via the cable 64. The electrical cable 64 can be led into the interior 3 of the chamber via a sealed passage 63 in a side wall 2s.

A nozzle 8 is also arranged in the interior 3. Said nozzle 8 has a nozzle body 81 and a circumferential nozzle wall 82 (see FIGS. 6 and 6A). The nozzle wall 82 encloses a channel 83 through which the test gas is guided to the first front face 81a of the nozzle body 81. A first nozzle opening 84 is formed on the first front face 81a through which the test gas can escape from the channel 83 and thus from the nozzle body 81 (arrow B). It can be seen in FIG. 6 that the nozzle body 81 tapers toward its first front face 81a. It can also be seen that the material thickness of the nozzle wall 82 decreases toward the first front face 81a. Thus, the nozzle body 81 has the shape of a tip. On the second front face 81b of the nozzle body 81 which is opposite the first front face 81a a window 85 is formed which enables observation of the held semiconductor component 102a by means of the optical device 9. The channel 83 extends from the second front face 81b to the first front face 81a of the nozzle body 81. The nozzle body 81 of the nozzle 8 is a symmetrical nozzle body with respect to the axis G which extends in the vertical direction (see FIGS. 6 and 6A). The longitudinal axis of the channel 83, the first front face 81a, the first nozzle opening 84 and the second front face 81b lie on the axis G. The term longitudinal axis refers to the z coordinate.

In addition, the nozzle 8 has a first supply channel 86 for guiding a gas into the channel 83 of the nozzle body 81 (see FIGS. 6 and 6C). In the first variant shown in FIG. 6, the test gas is guided through the first supply channel; in the second variant shown in FIG. 6C, the first gas is guided through the first supply channel 86. In the embodiment of the apparatus 1 according to the invention shown, the nozzle 8 also has a second supply channel 87 which in the second variant can serve to guide a second gas into the channel 83 of the nozzle body 81 or, if, as in the first variant, the supply of a second gas is not provided, is unused. The first supply channel 86 ends in an area of the nozzle wall 82 which is adjacent to the second front face 81b of the nozzle body 81. The second supply channel 87 also ends in an area of the nozzle wall 82 which is adjacent to the second front face 81b of the nozzle body 81. In the second variant, the first gas and the second gas mix in the channel 83 to form the test gas. In FIG. 6C, the first gas is referred to as “Gas 1” and the second gas is referred to as “Gas 2”. The test gas or the first gas is supplied to the nozzle 8 via a port 11 sealed by a seal 12 and a line 205 connected therethrough to the first supply channel 86. In the second variant, the second gas is supplied to the nozzle 8 via a port 14 sealed by means of a seal 15 and a line 16 connected therethrough to the first supply channel 86. However, as in the first variant, it is possible that no second gas is supplied. In this case, the second supply channel 87 is closed at port 14, as shown in the context of the first variant.

The two supply channels are formed in a circumferential flange 88. The flange 88 has an outer surface 88a which faces the holding surface 40 of the holding device 4. In the first embodiment of apparatus 1 according to the invention the nozzle 8 is supported with the outer surface 88a on the contacting device via connecting elements 10 (FIG. 7).

The nozzle body 81 extends through the opening 62 of the contacting device. Here, the first front face 81a of the nozzle body 81 and with it the first nozzle opening 84 is arranged opposite the holding surface 40 of the holding device 4. A distance D is formed between the first front face 81a of the nozzle body 81 and the holding device 4. The distance is selected such that the nozzle does not touch either the holding device 4 or a semiconductor component 102 when the substrate 101 is held by the holding device 4. The distance C between the front face 81a of the nozzle body 81 and the sensor layer 104 of the held semiconductor component 102a is less than distance D. Distance C should be in a range that is greater than 0 and less than 0.1 mm. The distance D corresponds to the sum of distance C and the thickness of the held semiconductor component 102a. The distances C and D and the thickness of the held semiconductor component 102a each refer to the z coordinate. This corresponds to the height direction.

The nozzle 8 is spaced apart from the holding surface 40 and, when the holding surface 40 holds a substrate 101, from the held semiconductor component 102a. For that, a gap 17 is formed between the first front face 81a of the nozzle body 81 and the semiconductor component 102a through which the test gas can flow away from the sensor layer 104 (arrow H). The gap 17 forms an overflow area. The size of the gap 17 is determined by the distance C and should be as small as possible.

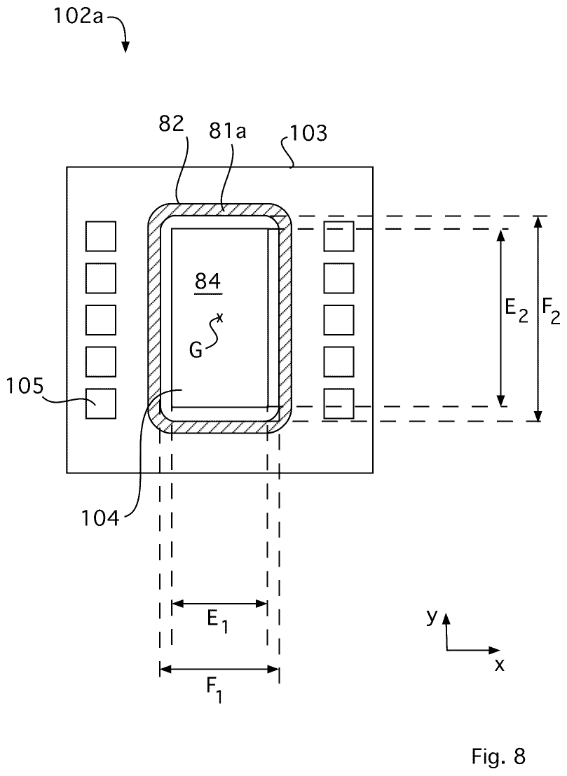

The first nozzle opening 84 lies opposite the sensor layer 104 of the held semiconductor component 102a and is spaced apart from it. The extension F1, F2 of the first nozzle opening in a plane parallel to the sensor layer 104 can correspond to the extension E1, E2 of the sensor layer 104 or be greater than the extension E1, E2 of the sensor layer 104 (see FIG. 8). The extension of the first nozzle opening 84 is such that on the one hand the test gas escaping through the nozzle opening 84 reaches either only the sensor layer 104 or only the sensor layer 104 and the surrounding area 107 and, on the other hand, the escaping test gas completely covers the sensor layer 104. As can be seen in FIG. 8 the geometric shape of the first nozzle opening 84 corresponds to the geometric shape of the sensor layer 104.

FIG. 8 shows the held semiconductor component 102a and the first front face 81a of the nozzle. Nozzle wall 82 which starts at the first front face 81a is shown hatched. As can be seen the extension F1, F2 of the first nozzle opening 84 is greater than the extension E1, E2 of the sensor layer 104. However, the extension F1, F2 of the first nozzle opening is less than the extension of the held semiconductor component 102a. The extension F1, F2 of the first nozzle opening corresponds to the extension E1, E2 of the sensor layer 104 plus a surrounding area 107. Thus, the nozzle opening does not extend over the contact surfaces 105. The extension E1 and the extension F1 refer to the x coordinate. The extension E2 and the extension F2 on the other hand refer to the y coordinate.

If no substrate is held by the holding device 4 the first nozzle opening 84 is opposite the first holding area 41 of the holding surface 40.

In chamber 2 there are formed sealed openings which enable the negative pressure or the positive pressure to be adjusted in the interior 3. In the first embodiment of the apparatus 1 according to the invention a device for adjusting a negative pressure is provided.

The apparatus 1 according to the invention has an inlet 18 via which the ambient gas which can be an inert gas can be supplied to the interior 3 of the chamber 2. The inlet 18 has a butterfly valve 181 and a cut-off valve 182 which both are arranged outside the chamber 2. In the chamber wall there is formed a sealed opening 183 through which the ambient gas can get out of the inlet 18 in the interior 3 (arrow ZL). The inlet 18 has a pipe or a sequence of pipes. The opening 183 is formed in the bottom 2u of the chamber 2.

The apparatus 1 according to the invention further has an outlet 19 via which a negative pressure can be adjusted and maintained in the interior 3. The outlet has a pump 191 and a control valve 192 which both are arranged outside the chamber 2. In the chamber wall there is formed a sealed opening 193 through which gas can be withdrawn from the interior 3 in the outlet 19 by means of the pump 191 (arrow AL). The outlet 19 has a pipe or a sequence of pipes. The opening 193 is formed in the bottom 2u of the chamber 2.

The apparatus 1 according to the invention additionally has a supply 20 for the test gas. The supply 20 has a line or a sequence of lines. The supply 20 has a butterfly valve 201 and a cut-off valve 202 which both are arranged outside the chamber 2. In the chamber wall there is formed a sealed passage 203 for a line of supply 20. The first gas (“Gas 1”) is supplied to the first supply channel 86 via a line of supply 20. The passage 203 is formed in a side wall 2s of the chamber 2.

When a second gas (Gas 2) is supplied to the nozzle the apparatus 1 according to the invention has a second supply the construction of which can correspond to supply 20 for gas 1, with the exception that gas 2 is supplied to the second supply channel 87. Line 16 is part of the second supply.

The apparatus 1 according to the invention can have a measuring device 21. The measuring device 21 in the first embodiment of the apparatus 1 according to the invention shown is arranged in the interior 3 of chamber 2. However, it can also be arranged outside the chamber 2. The measuring device 21 can be used to determine process parameters. For example, the measuring device 21 can be employed to determine the concentration of the test gas or one of its components, the pressure in the interior 3, the flow rate, and/or the volume stream of the test gas or a component of the test gas. The measuring device 21 can be integrated into the supply 20, as is shown in FIG. 3.

A door 22 which can serve as a loading door is formed on a side wall 2s. The contact surfaces between the side wall 2s on which the door 22 is formed and the door 22 are sealed by means of one or more sealing elements. The door provides access to the interior 3. Windows 23 may be formed in one or more side walls 2s wherein the contact surfaces between the side wall 2s in which the window 23 is formed and the window 23 are sealed by means of one or more sealing elements. In the first embodiment of the apparatus 1 according to the invention shown in FIG. 3 only one window 23 is provided. The window(s) 23 make it possible to observe the interior 3, the devices located there and the substrate 101 with the semiconductor component 102.

A central opening 24 is formed in the lid of the chamber 2 which is closed by means of an insert 25 wherein the contact surfaces between the insert 25 and the lid are sealed by means of one or more sealing elements. The insert has a sealed window 26 through which observation of the semiconductor component 102 is possible by means of the optical device 9.

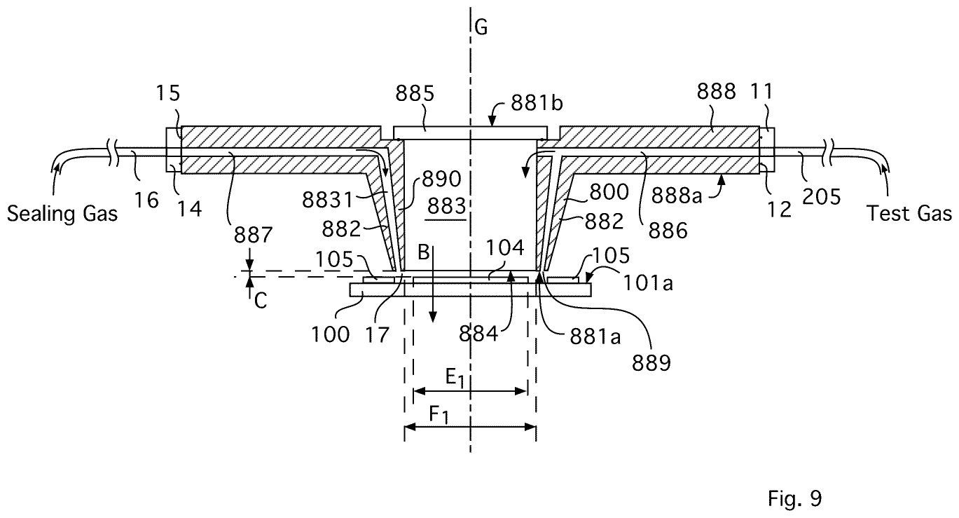

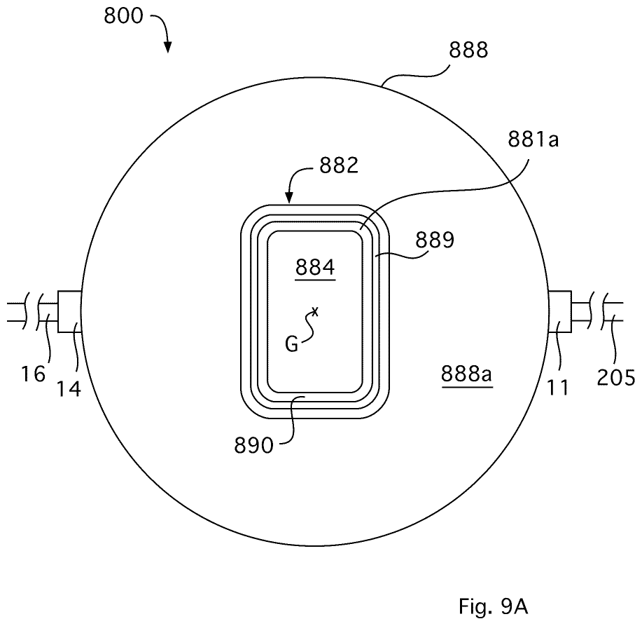

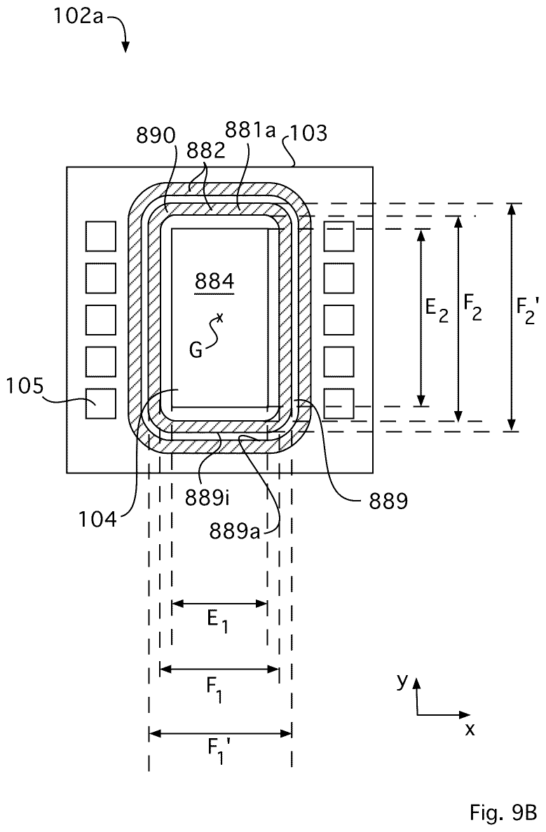

FIGS. 9 to 11 show a second nozzle 800 the arrangement and orientation of which in interior 3 corresponds to the arrangement of nozzle 8. The construction of the second nozzle 800 corresponds to the construction of the nozzle 8 apart from a second nozzle opening 889 and the changes to the nozzle 800 associated with this nozzle opening 889 compared to the nozzle 8. The second nozzle opening 889 and the associated changes are described below.

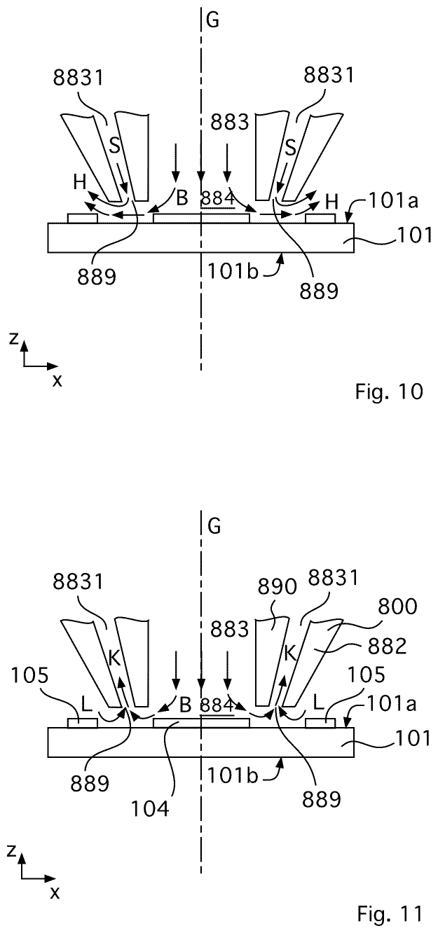

The nozzle 800 has a nozzle body 881 and a circumferential nozzle wall 882 (see FIG. 9). The nozzle wall 882 encloses a first channel 883 through which the test gas is guided to the first front side 881a of the nozzle body 881, and a second channel 8831. A sealing gas is guided through the second channel 8831 to the first front face 881a of the nozzle body 881 (see FIG. 10) or the test gas is led off from the first front face 881a (see FIG. 11). A first nozzle opening 884 is formed on the first front face 881a through which the test gas can escape from the first channel 883 and thus from the nozzle body 881 (arrow B). A second nozzle opening 889 is also formed on the first front face 881a through which the sealing gas can escape from the second channel 8831 and thus from the nozzle body 881 (FIG. 10) or the test gas can enter the nozzle body again, namely into the channel 8831 (FIG. 11). The first nozzle opening 884 and the second nozzle opening 889 are separated from each other by a nozzle web 890 which also separates the first channel 883 from the second channel 8831.

As with nozzle 8 the nozzle body 881 of the second nozzle 800 tapers towards its first front face 881a. The material thickness of the nozzle wall 882 also decreases towards the first front face 881a. The nozzle body 881 thus also has the shape of a tip. On the second front face 881b of the nozzle body 881 which is opposite the first front face 881a a window 885 is formed which enables observation of the held semiconductor component 102a by means of the optical device 9. The first channel 883 extends from the second front face 881b to the first front face 881a of the nozzle body 881. As to its outer shape the nozzle body 881 of the nozzle 800 is a symmetrical nozzle body relative to the axis G which extends in the height direction (see FIG. 9). The longitudinal axis of the first channel 883, the first front face 881a, the first nozzle opening 884, the second nozzle opening 889 and the second front face 881b lie on the axis G. The term longitudinal axis refers to the z coordinate.

The second nozzle opening 889 extends around the first nozzle opening spaced apart by the nozzle web 890 (see FIG. 9A). It is coaxial with the first nozzle opening. The edge of the second nozzle opening 889 facing the first nozzle opening 884 is the inner edge 889i of the second nozzle opening. The edge of the second nozzle opening 889 facing the first nozzle opening 884 is the outer edge 889a of the second nozzle opening. The geometric shape of the inner edge 889i corresponds to the geometric shape of the edge of the first nozzle opening, but the extension of the inner edge 889i along the x and y coordinates is larger, whereby forming the nozzle web 890. The geometric shape of the outer edge 889a corresponds to the geometric shape of the inner edge 889i of the second nozzle opening, but the extension of the outer edge 889a along the x and y coordinates is greater than that of the inner edge 889i.

Like nozzle 8 the second nozzle 800 has a first supply channel 886 for guiding the process gas into the first channel 883 of the nozzle body 881. The nozzle 800 also has a second supply or discharge channel 887 which in a first variant can serve to guide a second gas from the second channel 8831 of the nozzle body 881—in which case the second supply or discharge channel 887 is a discharge channel—or which in a second variant can serve to guide the test gas into the second channel 8831 of the nozzle body 881—in which case the second supply or discharge channel 887 is a supply channel. The first supply channel 886 ends in an area of the nozzle wall 882 which is adjacent to the second front face 881b of the nozzle body 881. The second supply or discharge channel 887 also ends in an area of the nozzle wall 882 which is adjacent to the second front face 881b of the nozzle body 881. In the first variant (FIG. 10) and in the second variant (FIG. 11) the test gas is supplied to the nozzle 800 via a port 11 sealed by means of a seal 12 and a line 205 connected therethrough to the first supply channel 886. In the first variant the sealing gas is supplied to the nozzle 800 via a port 14 sealed by means of a seal 15 and a line 16 connected therethrough to the second supply or discharge channel 887. In the second variant the test gas is discharged from the nozzle 800 via a port 14 sealed by means of a seal 15 and a line 16 connected therethrough to the second supply or discharge channel 887. An exhaust pump can be provided for this purpose.

The first supply channel 886 and the second supply or discharge channel 887 are formed in a circumferential flange 888. The flange 888 has an outer surface 888a facing the holding surface 40 of the holding device 4. In the first embodiment of the apparatus 1 according to the invention the second nozzle 800 is supported with the outer surface 888a on the contacting device via connecting elements 10, as shown in the context of nozzle 8 in FIG. 7.

The nozzle body 881 extends through the opening 62 of the contacting device. The first front face 881a of the nozzle body 881 and with it the first nozzle opening 884 and the second nozzle opening 889 are arranged opposite the holding surface 40 of the holding device 4. A distance D is formed between the first front face 881a of the nozzle body 881 and the holding device 4, as shown in the context of nozzle 8 in FIG. 6B. The distance is selected such that the nozzle 800 does not touch either the holding device 4 or a semiconductor component 102 when the substrate 101 is held by the holding device 4. The distance C between the front face 881a of the nozzle body 881 and the sensor layer 104 of the held semiconductor component 102a is less than the distance D. Distance C should also be in a range that is greater than 0 and less than 0.1 mm. The distance D corresponds to the sum of distance C and the thickness of the held semiconductor component 102a. Distances C and D as well as the thickness of the held semiconductor component 102a each refer to the z coordinate. This corresponds to the height direction.

The nozzle 800 is spaced apart from the holding surface 40 and when the holding surface 40 holds a substrate 101 from the held semiconductor component 102a. For that, a gap 17 is formed between the first front face 881a of the nozzle body 881 and the semiconductor component 102a. The size of the gap 17 is determined by the distance C and should be as small as possible. In the first variant the test gas-which has entered the gap 17 through the first nozzle opening (arrow B)—and the sealing gas—which has entered the gap 17 through the second nozzle opening (arrow S)—can flow off through the gap 17 (arrow H in FIG. 10). Then, the gap 17 forms an overflow area. In the second variant the test gas after having escaped from the first nozzle opening 884 and entered the gap 17 (arrow B in FIG. 11) again enters the nozzle 800 via the second nozzle opening 889 (arrow K) whereby ambient gas from the interior 3 can also enter the nozzle 800 via the second nozzle opening 889 (arrow L).

The first nozzle opening 884 and the second nozzle opening 889 are located opposite the sensor layer 104 of the held semiconductor component 102a and are spaced apart from it. FIG. 9B shows the held semiconductor component 102a and the first front face 881a of the nozzle 800. The nozzle wall 882 which starts at the first front face 881a is shown hatched including the nozzle web 890. As can be seen, the extension F1, F2 of the first nozzle opening 884 is greater than the extension E1, E2 of the sensor layer 104. The extension F1′, F2′ of the inner edge 889i of the second nozzle opening 889 is greater than the extension F1, F2 of the first nozzle opening. However, the extension F1, F2 of the first nozzle opening is less than the extension of the held semiconductor component 102a. The extension F1, F2 of the first nozzle opening corre-sponds to the extension E1, E2 of the sensor layer 104 plus a circumferential area 107. Thus, the first nozzle opening 884 and the second nozzle opening 889 do not extend over the contact surfaces 105. Extension E1, extension F1, and extension F1′ refer to the x coordinate. Extension E2, extension F2, and extension F2′ on the other hand refer to the y coordinate.

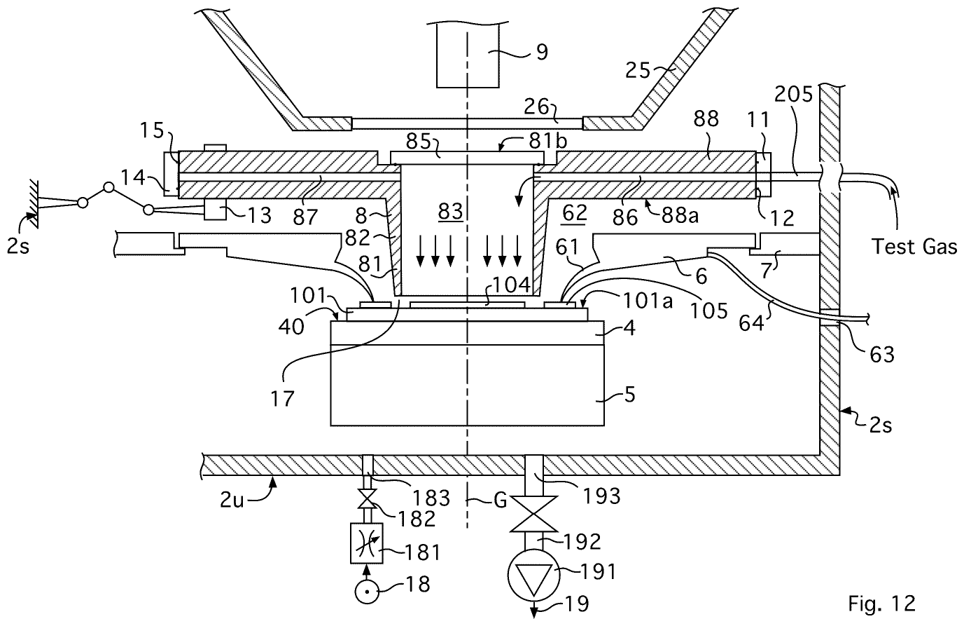

The second embodiment of an apparatus according to the invention shown in FIG. 12 corresponds to the first embodiment, except that the nozzle 8 is not attached to the contacting device 6. For that, no connecting elements 10 are required. In the second embodiment a positioning device 13 is provided instead with which the nozzle 8 can be moved in the x, y and z directions and can be rotated about an axis lying on the z coordinate. By means of the positioning device 13 the nozzle 8 can be positioned such that the first front face is opposite the holding surface 40 at the predetermined distance D or at the predetermined distance C from the sensor layer 104 of the held semiconductor component, as shown in FIG. 8. The positioning device 13 can be attached to the side wall 2s of the chamber 2.

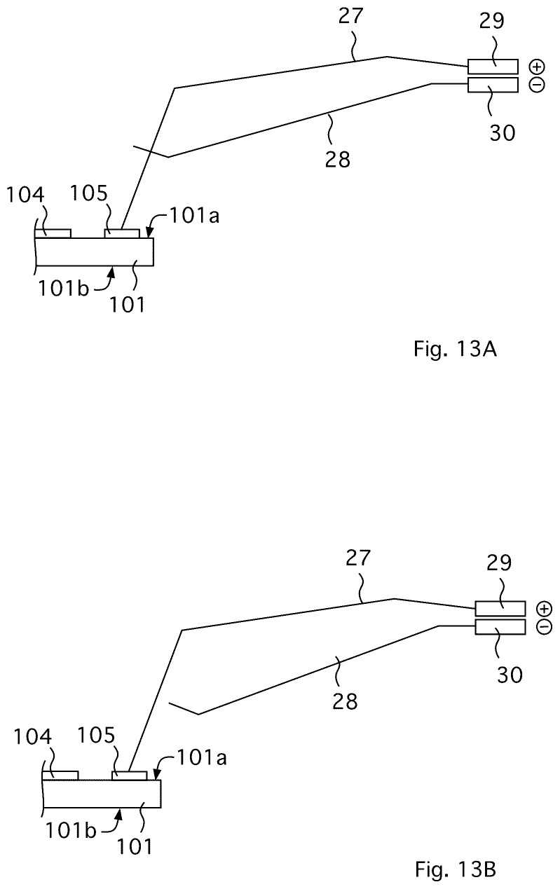

FIGS. 13A and 13B show an embodiment of a device for measuring the distance between the held semiconductor component 102a and the nozzle 8. This measuring device is attached to the nozzle 8 (not shown) and has a test needle 27 and a contact element 28. The test needle 27 is crossed with the contact element 28 (see FIG. 13A). The contact element 28 can also be needle-shaped. At the crossing point the test needle 27 and the contact element 28 touch each other to form an electrical connection. The test needle 27 which is connected to port 29 and the contact element 28 which is connected to port 30 belong to a circuit. The test needle 27 has a tip which-with respect to the main body of the test needle-extends obliquely in the direction of the held semiconductor component 102a, for example at an angle of 45° to its main body. If the nozzle and with it the test needle 27 is moved towards the held semiconductor component 102a the tip comes into contact with a contact surface 105 which is formed on the held semiconductor component 102a for this purpose.

FIG. 13A shows the initial state. In this state, the tip of the test needle 27 does not touch the contact surface 105. The distance between the nozzle and the held semiconductor component is large. If now the nozzle is moved along the z coordinate on the axis G towards the held semiconductor component 102a to reduce the distance the tip of the test needle 27 comes into contact with the contact surface 105 (see FIG. 13B). As soon as the tip touches the contact surface the contact between the test needle 27 and the contact element 28 ends at the crossing point. The angled tip of the test needle 27 bends as soon as it touches the contact surface. In doing so, it performs a sliding movement along the x and/or z coordinate. The contact element 28 no longer touches the needle, so that the electrical connection between the test needle 27 and the contact element 28 ends. The circuit is interrupted. This interruption can be detected, for example with software. Then, the movement of the nozzle along the z coordinate is stopped.

The sliding movement is clearly visible from above using the optical device 9, for example a microscope. In contrast to the previously problematic estimation of the height distance, this sideways movement is a very good indication that contact has been made between the test needle 27 and the held semiconductor component.

### LIST OF REFERENCE SYMBOLS

1 apparatus
2 chamber
2o ceiling
2s side wall
2u bottom
3 interior
4 holding device
40 holding surface
41 first holding area
42 second holding area
43 third holding area
5 positioning device
6 contacting device
61 contacting needle
62 opening
63 passage
64 electrical cable
7 holder
8 nozzle
81 nozzle body
81a first front face of the nozzle body
81b second front face of the nozzle body
82 nozzle wall
83 channel
84 first nozzle opening
85 window
86 first supply channel
87 second supply channel
88 flange
88a outer surface
9 optical device
10 connecting element
11 port
12 sealing
13 positioning device
14 port
15 sealing
16 line
17 gap
18 inlet
181 butterfly valve
182 cut-off valve
183 opening
19 outlet
191 pump
192 control valve
193 opening
20 supply
201 butterfly valve
202 cut-off valve
203 passage
205 line
21 measuring device
22 door
23 window
24 opening
25 insert
26 window
27 test needle
28 contact element
29 port
30 port
800 second nozzle
881 nozzle body
881a first front face of the nozzle body
881b second front face of the nozzle body
882 nozzle wall
883 first channel
8831 second channel
884 first nozzle opening
885 window
886 first supply channel
887 second supply or discharge channel
888 flange
888a outer surface
889 second nozzle opening
889i inner edge
889a outer edge
890 nozzle web
101 substrate
101a top side of the substrate
101b bottom side of the substrate
102 semiconductor component
102a exemplary semiconductor component
103 boundary of semiconductor component
104 sensor layer
105 contact surface
106 spacer surface
106a spacer surface
107 circumferential area

1-15. (canceled)

16. An apparatus for testing an electronic semiconductor component which as a sensor layer for detecting a gaseous substance and is formed on a substrate using a test gas, wherein the apparatus has a chamber enclosing an interior in which negative or positive pressure prevails and in which there are arranged
a holding device for holding the substrate;
a nozzle for directionally delivering the test gas onto the sensor layer of the electronic semiconductor component formed on the substrate held by the holding device; and
a contacting device for electrically contacting the semiconductor component at its contact surface, wherein the contacting device is spaced apart from the nozzle;
wherein the contacting device has an opening through which the nozzle is passed and wherein the nozzle has a nozzle body with a nozzle wall which is spaced apart from the opening in the contacting device.

17. The apparatus according to claim 16, wherein the nozzle is configured to deliver a sealing gas or to receive the test gas.

18. The apparatus according to claim 16, wherein the nozzle has a nozzle body having a first nozzle opening, wherein the first nozzle opening is arranged opposite the sensor layer.

19. The apparatus according to claim 18, wherein the first nozzle opening is arranged in alignment with the sensor layer.

20. The apparatus according to claim 18, wherein the nozzle body has a first front side in which the first nozzle opening is formed, wherein the distance of the first front side of the nozzle body from the sensor layer of the electronic semiconductor component is greater than 0 and smaller than 0.1 mm.

21. The apparatus according to claim 20, wherein the nozzle body has a channel extending to the first front side in which the first nozzle opening is formed.

22. The apparatus according to claim 17, wherein the nozzle has a second nozzle opening for delivering the sealing gas or for receiving the test gas, wherein the second nozzle opening is form within the nozzle body of the nozzle.

23. The apparatus according to claim 22, wherein the nozzle opening is formed circumferentially around the first nozzle opening to form a nozzle web.

24. The apparatus according to claim 22, wherein the electronic semiconductor component has a contact surface which is spaced apart from the sensor layer to form a spacer surface, wherein the second nozzle opening is opposite the spacer surface.

25. The apparatus according to claim 16, wherein the nozzle tapers toward the holding device.

26. The apparatus according to claim 16, wherein the nozzle has a nozzle wall the material thickness of which decreases toward the holding device.

27. The apparatus according to claim 16, further comprising a device for displaying the distance and/or for measuring the distance between the sensor layer and the nozzle.

28. The apparatus according to claim 16, further comprising a positioning device for positioning the nozzle.

29. The apparatus according to claim 16, wherein the nozzle is connected to the contacting device.

30. A method for testing an electronic semiconductor component which has a sensor layer for detecting a gaseous substance and is formed on a substrate using a test gas by an apparatus which has a chamber enclosing an interior in which negative or positive pressure prevails, comprising
positioning a nozzle with respect to the sensor layer of the electronic semiconductor component to enable a directional delivery of the test gas onto the sensor layer, wherein the nozzle is passed through an opening of a contacting device;
contacting contact surfaces of the electronic semiconductor component by a contacting device; and
outputting a test signal at contact surfaces of the electronic semiconductor component by the contacting device while the test gas is delivered to the sensor layer by a nozzle.
